(19) United States
(12) Patent Application Publication
(10) Pub. No.: US 2024/0268803 A1
(43) Pub. Date: Aug. 15, 2024

(54) SUPPORT TECHNIQUE AND PLACEMENT OF REABSORBABLE THREADS IN THE SUBURETHRAL SPACE, FOR THE NON-SURGICAL TREATMENT OF GRADE 3 STRESS URINARY INCONTINENCE IN WOMEN, AND INSTRUMENTS FOR ITS PRACTICE

(71) Applicant: ARIEL LUKSENBURG WIERNIK, MONTEVIDEO (UY)

(72) Inventor: ARIEL LUKSENBURG WIERNIK, MONTEVIDEO (UY)

(21) Appl. No.: 18/139,982

(22) Filed: Apr. 27, 2023

(30) Foreign Application Priority Data
Apr. 27, 2022 (UY) 39739

(51) Int. Cl. A61B 17/00

(52) U.S. Cl. CPC A61B 17/00; A61B 2017/00004; A61B 2017/00805

(57) ABSTRACT

A technique for the treatment of stress urinary incontinence in women, characterized by the placement of a network of absorbable threads in the sub urethral and urethro vaginal spaces, and instruments for its execution. The technique includes the placement of absorbable polydioxanone or polycaprolactone threads in the non-surgical and minimally invasive treatment of grade 3 stress urinary incontinence in women.

### CROSS REFERENCE TO RELATED APPLICATION

This application claims the benefit of and takes priority from Uruguay Patent Application No. 39739 filed on Apr. 27, 2022, the contents of which are herein incorporated by reference.

### BACKGROUND OF THE INVENTION

### Field of the Invention

The novel technique claimed as patentable includes the placement of absorbable polydioxanone or polycaprolactone threads in the non-surgical and minimally invasive treatment of grade 3 stress urinary incontinence in women.

It seeks to tighten and restore support to the suburethral and vaginal urethral spaces, the anatomical site through which the urethra runs and supports on its way from the bladder to the urethral meatus. The weakening or flaccidity of these anatomical spaces caused by childbirth or hormonal deficit-among others-, determines the so-called “urethral hypermotility”, one of the main known causes responsible for true stress urinary incontinence.

### DESCRIPTION OF THE RELATED ART

Currently, the surgical technique that is applied in cases of true urine incontinence is the technique called Trans Obturatriz Type (TOT), consisting of the application and placement of a mesh of non-resorbable material below the urethra, which thus rectifies way your journey. Said mesh also determines a reaction of the organism, which usually forms a fibrosis around it, as a reaction of the organism to such a foreign body.

The surgical procedure described was the “gold standard” for many years. However, due to the complications and its relative efficacy, after more than 20 years it has been discouraged by several countries with the result that in many of them they return to the previous technique—the modified Burch surgery-which implies a surgical activity of greater complexity and with a longer postoperative period.

### SUMMARY OF THE INVENTION

The novel technique that is claimed is aimed at those patients who present severe urinary incontinence, grade (with minimal efforts), who until now only knew of a single solution: the surgical option. It consists of placing a network of absorbable threads of different types in the suburethral and urethral-vaginal spaces (lateral to the urethra), forming a crisscross network in the latter (sub urethral spyder web), and another on both sides of the urethra, in the urethrovaginal space. In this way, a mesh of absorbable threads is completed, from ischio-pubic branch to branch, placed throughout the urethrovaginal space.

This innovative technique has a double mechanism of action; one immediate and one in the medium and long term. The urethra is immediately provided with a new physical support, in all its extension (approximately 3 cms.), with the placement of a network of absorbable threads in the suburethral space. In the same way as the surgical technique, the threads will generate a foreign body reaction and trigger a fibrosis reaction around it, thereby enhancing—in the medium and long term—the support effect that is generated when the network of threads is placed. The great advantage of this technique is that it is an office procedure, ambulatory, painless, without post-operative and with immediate return to patient's job.

For its execution, the claimed technique foresees the use of a series of instruments—also novel—that facilitate and guide the procedure in a safe way and avoiding errors and iatrogenesis, as well as the use of specific inputs. They are:

### Polyprolactone (PCL) or Polydioxanone (Pdo) Threads:

PDO and PCL threads are very popular in the aesthetic area. These are absorbable threads that are mounted inside a blunt-tipped needle or cannula, protruding from the tip to retract in the opposite direction, and externally attached to the same needle or cannula to be held in place by a polyurethane material.

For the execution of the technique described, the threads are of special measures (according to the anatomy of the urethra and the urethral-vaginal space), and their application consists of introducing them by means of their assembly in suitable instruments or tools—also novel that will be described.

The application of these threads is similar for the different sizes and models. For their application, they are introduced through the urethral-vaginal space by mounting them on the aforementioned tools, and once the needle or cannula accesses the desired anatomical site (sub-urethral and lateral spaces to the urethra) they are withdrawn with the result that when coming into contact with the fabrics, the threads remain located in the same place in which they were originally introduced.

### I).—the Mesh Threads:

The mesh threads are made of six 6.0 threads, intertwined and introduced into a 19G 38 mm cannula, with a blunt tip to avoid injuries during the trajectory of its use. For its application, it is necessary to make an entry hole with a needle.

### II).—the Pluri Threads:

Pluri threads are developed by five threads. Like the mesh threads, they are presented inside a 21G 38 mm blunt cannula and its terminal ends with a sort of “horsetail”. As in the previous case, its use requires previously making an entry hole with a suitable needle.

### III).—the Mono Screw Threads:

They are simple turned threads (MONOSCREW 26 g 30 MM) that are mounted on a sharp needle.

### C).—the Tools:

### I).—the Fenestrated Speculum 3

Made of stainless steel, it has two lateral and longitudinal fenestrations or “windows”, 7 mm wide by 65 mm long. In its center—and between both fenestrations or windows—it presents a central and longitudinal area (“central nerve”), whose function is to provide stability to the speculum and mechanical protection to the urethra during the procedure. On the inner edges of each window there are four inclined slots, and the function of these “windows” is to guide the operator during the procedure, guiding and delimiting the area where the needles must be inserted with the Monoscrew wire mounted on the needles. In turn, the inclined grooves will guide the technician in the application of the Pluri threads.

It also has a notch at 12 o'clock, which acts as a support when using the LS3 Introducer, thereby protecting the urethra when threads are applied to the urethra or close to the urethra.

### II).—the Introducer 3:

The introducer 3 is an innovative tool made of stainless steel, with the measurements and shape described in FIG. 5A, which is always used covered from its distal end, with a sterile bladder catheter no. 18 (FIG. 5C). With its handle attached and up, it enters the urethra through the urethral meatus

(perpendicular to it), and is inserted through the entire urethra to the bladder. Once located, it rises toward the zenith and deploys the vulvar vestibule, elevating the urethra to unfold the suburethral space, thereby providing safety and protection to the urethra during placement of strings in the suburethral space.

### III).—the Bayonet No. 1:

It is a tool developed for the intravaginal application of threads. It represents an “extension of the technician's hand” that will allow you to easily maneuver inside the vagina when placing the threads on the front side of the vagina.

It measures 23.5 cm. in its full extension, and has a 10 cm long handle and a 13.5 cm long rod; shaped like an “italic S” and with an angle of 30°. Ends with a pointer adaptable to pressure to the back of the needle or cannula that carries the thread.

This instrument is used to place pluri wires and monoscrew wires. For the former, once the cannula is placed on the pointer of the bayonet 1, and following the path of the inclined slots (4) of the speculum windows, it is applied to the anterior face of the vagina with the speculum located at the 12th hour. It enters at an angle of 20° for a path of 1 cm and then rectifies its path until its total introduction is complete.

The same tool is also used to place the monoscrew threads with needles and in the urethrovaginal space, laterally to the urethra. Once the needle with the monoscrew thread is placed in the pointer of bayonet 1, the speculum is rotated 1 cm to the right and through its right window, and 3 threads are applied following the longitudinal axis of the speculum.

The application of the threads on the anterior face of the vagina is obtained by entering at an angle of 20° for a distance of 1 cm and then, to later rectify its path until full introduction. This done, the speculum is rotated two more times to the right, in the direction of the right ischio-pubic ramus, and three more threads are placed at each rotation. The maneuver is then repeated to the left.

### IV).—the Thread Holder:

This solid stainless steel tool has a cylindrical body 4 cm long by 1.8 cm in diameter, and an adaptable pressure pointer where the mesh threads will be placed for placement (FIGS. 7A, 7B and 7C).

### BRIEF DESCRIPTION OF THE DRAWINGS

FIG. 1A illustrates a mesh thread mounted to a blunt tipped needle or cannula.

FIG. 1B illustrates an enlarged view of the mesh thread.

FIG. 1C illustrates an enlarged view of the mesh thread in comparison to the blunt tipped needle.

FIG. 2 illustrates a set of five threads (pluri threads) to be mounted on the cannula.

FIG. 3A illustrates a mono screw thread.

FIG. 3B illustrates an enlarged view of the mono screw thread.

FIG. 4A illustrates a speculum with a pair of lateral fenestrations (windows).

FIG. 4B illustrates the lateral fenestrations.

FIG. 4C illustrates another view of the lateral fenestrations.

FIG. 4D illustrates an LS3 introducer.

FIG. 5A illustrates an introducer made of stainless steel for its introduction to the urethra.

FIG. 5B illustrates another view of the introducer.

FIG. 5C illustrates the introducer with a bladder catheter.

FIG. 6A illustrates a tool for the intravaginal application of threads.

FIG. 6B illustrates another view of the tool for the intravaginal application of threads.

FIG. 7A illustrates a tool to hold threads which a pressure pointer.

FIG. 7B illustrates another view of the tool to hold threads.

FIG. 7C illustrates the tool for thread holder in use.

FIGS. 8A-8B illustrate the introducer being placed through the urethral meatus.

FIGS. 8C-8D illustrate the introducer being raised to allow elevation of the urethral meatus and lengthening of the vulvar vestibule.

FIG. 9A illustrates a flaccid urethra.

FIG. 9B illustrates the urethra catheterized with the introducer.

FIG. 9C illustrates the suburethral space to allow access and placement of the mesh threads.

FIG. 10 illustrates a pair of entry holes in the vestibule.

FIGS. 11A-11B illustrate a method for the introduction of the mesh thread through one of the entry holes.

FIGS. 11C-11D illustrate placing and crossing the mesh thread in the suburethral space.

FIG. 12A illustrates placement of the speculum.

FIG. 12B illustrates the speculum in a rotated position and the introducer fitted into the speculum notch.

FIG. 13A illustrates the front face of the speculum.

FIG. 13B illustrates an assembled pluri thread.

FIG. 13C illustrates the placement of the bayonet in the suburethral space.

FIG. 14 illustrates a view of the network of threads placed in the suburethral space from above.

FIG. 15A illustrates the speculum windows with the bayonet.

FIG. 15B illustrates the mono screw threads.

FIG. 15C illustrates the speculum in a different position entering the urethrovaginal space.

FIG. 15D illustrates the placement of the threads by rotating the speculum.

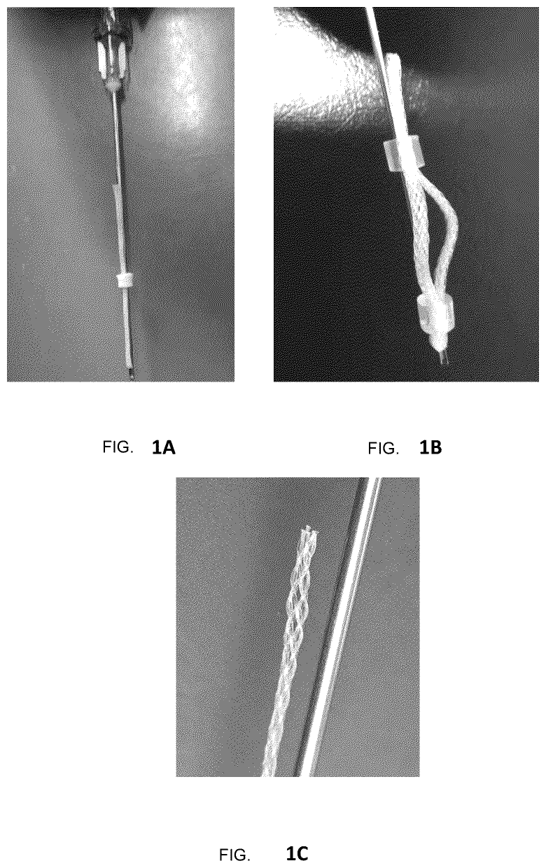

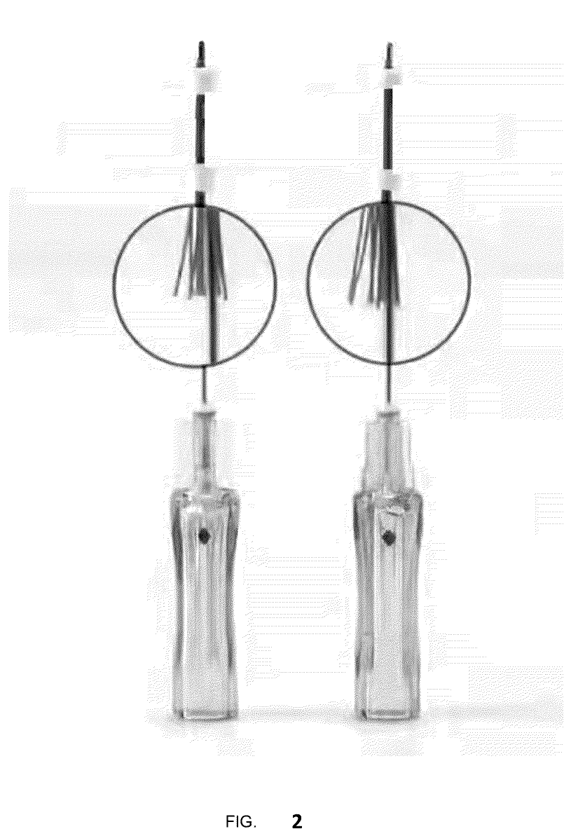

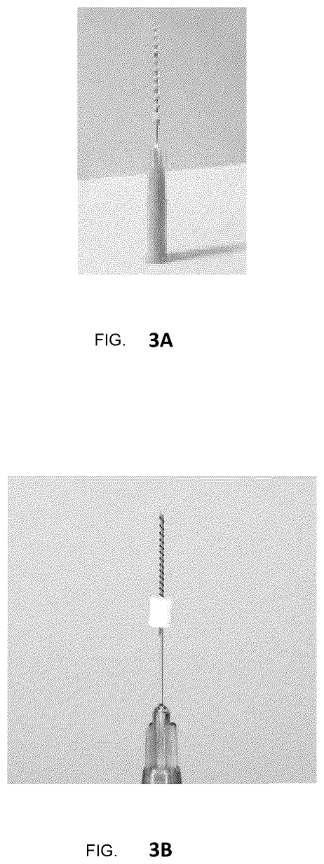

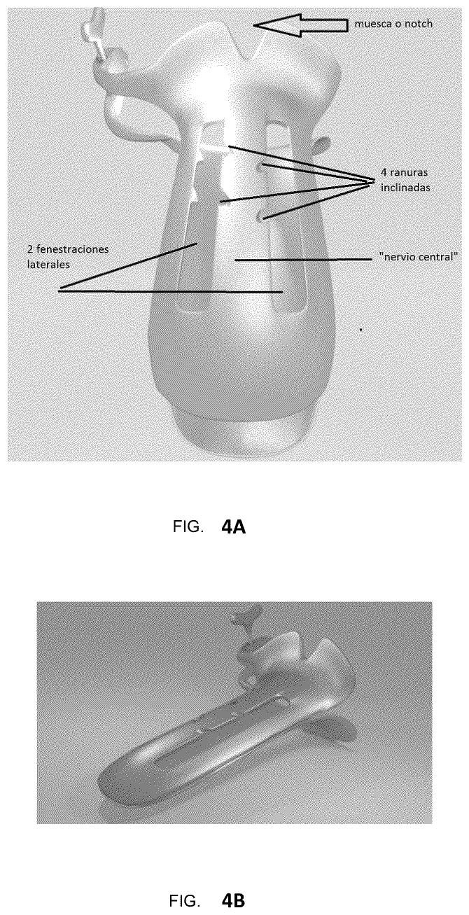

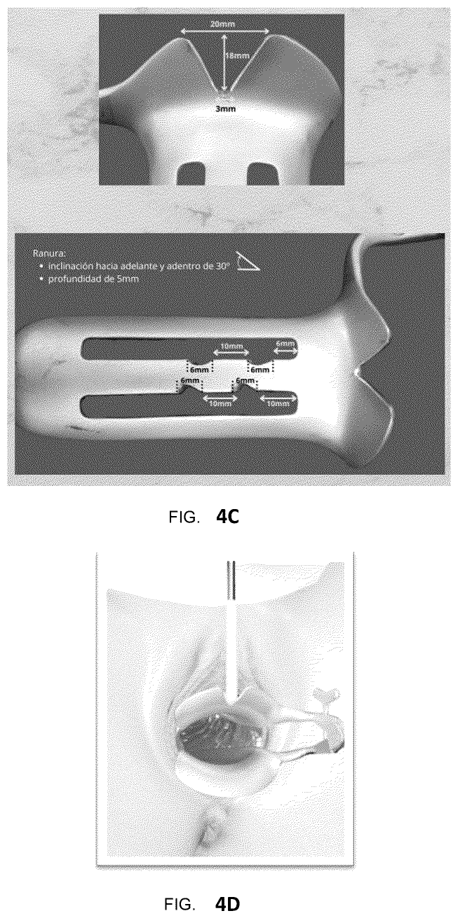

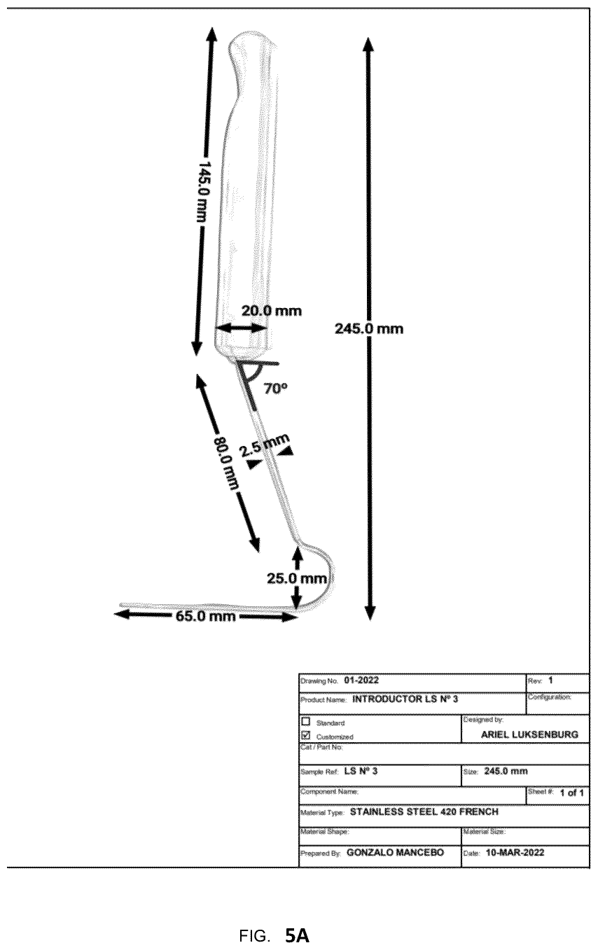

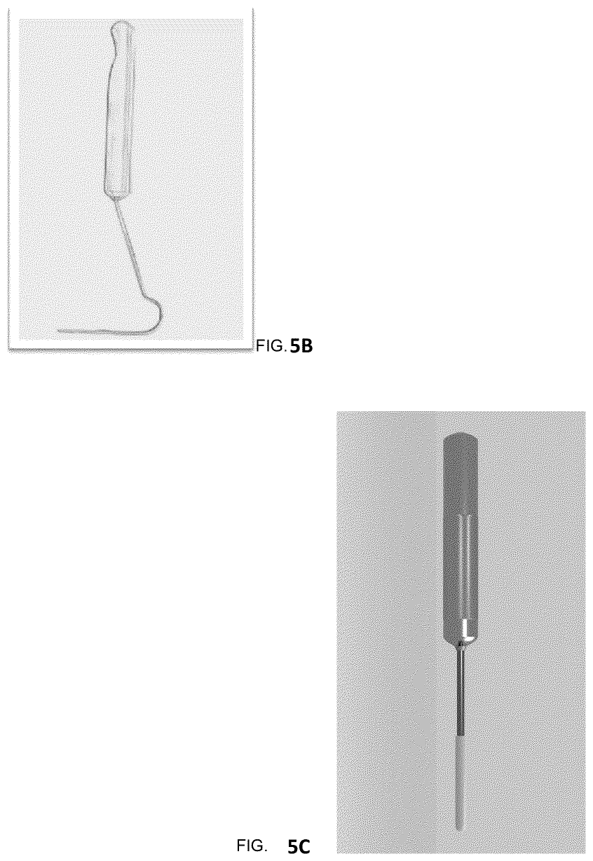

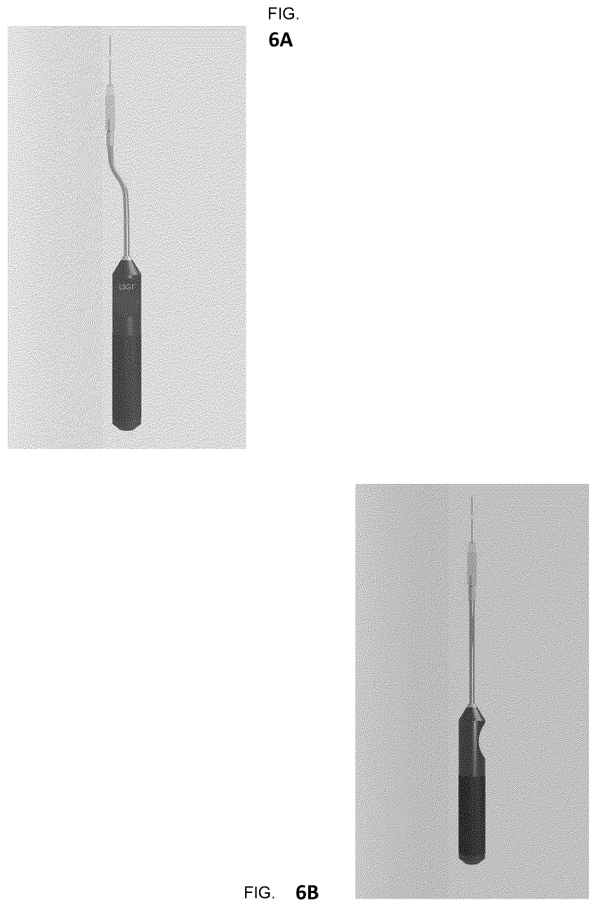

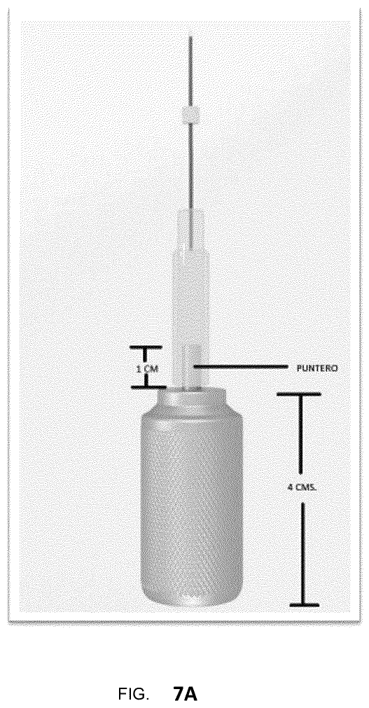

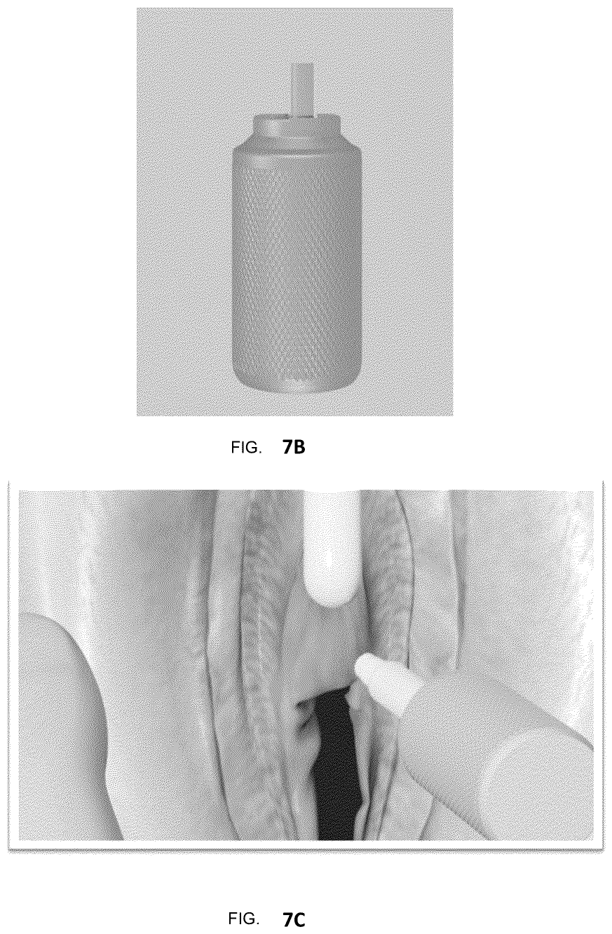

### D).—DESCRIPTION OF THE TECHNIQUE

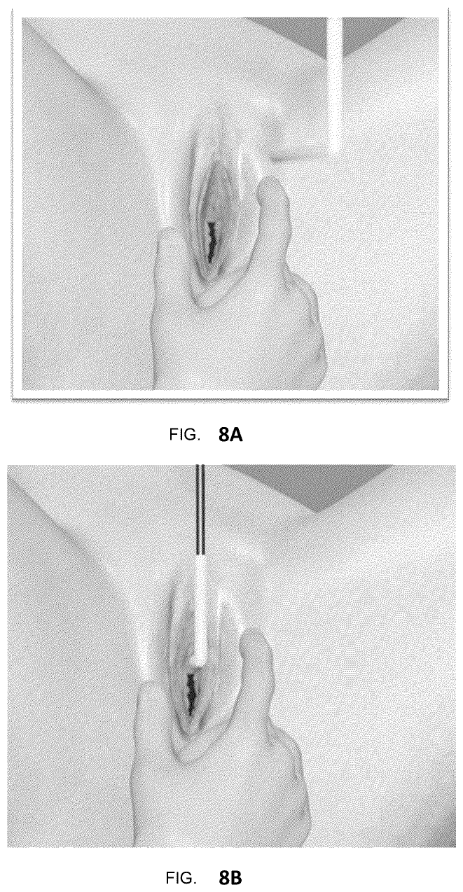

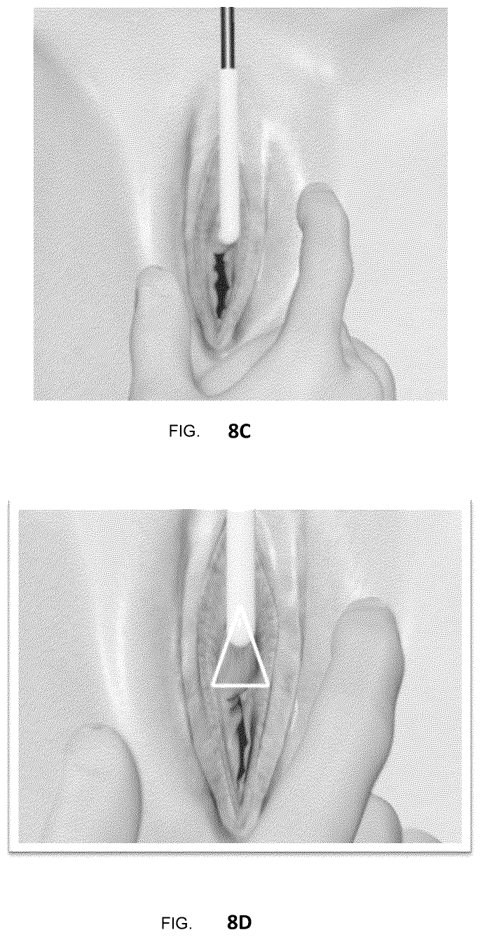

After supplying topical anesthesia to the vulva and vagina, the introducer is placed through the urethral meatus (FIGS. 8A and 8B), and then it is raised to allow elevation of the urethral meatus and lengthening of the vulvar vestibule (FIG. 8D).

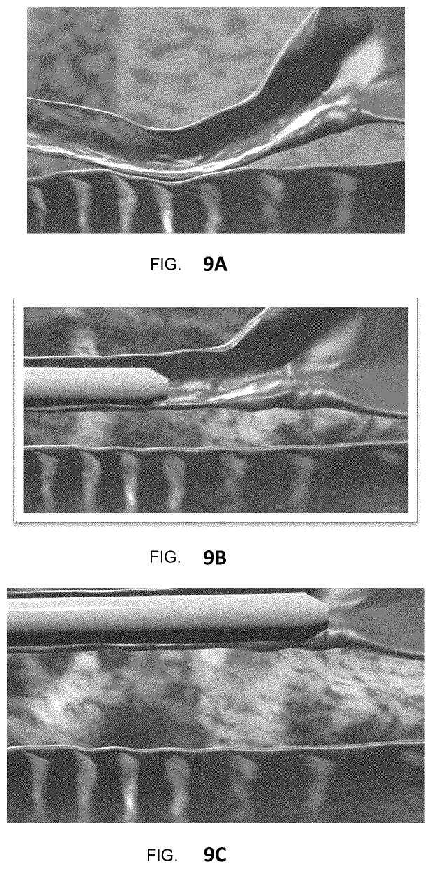

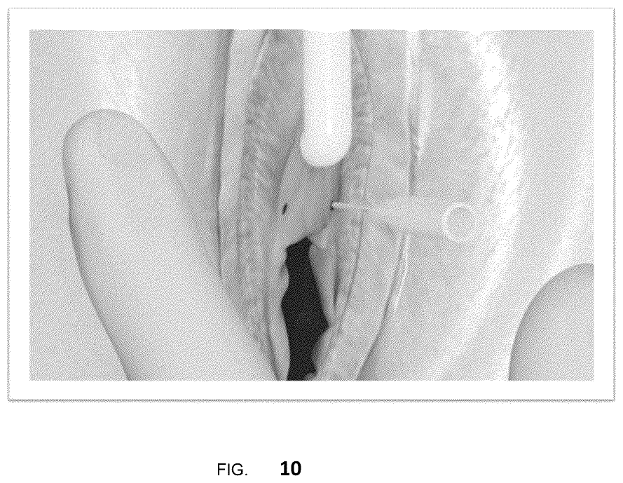

Subsequently, the urethra is catheterized with Introducer 3, with a mounted catheter, in bladder resulting its rectification and elevation (FIG. 9B). In this way, when the urethra is raised, the suburethral space will be enlarged and will allow access and placement of the mesh or network of threads without risk of injury to the urethra (FIG. 9C).

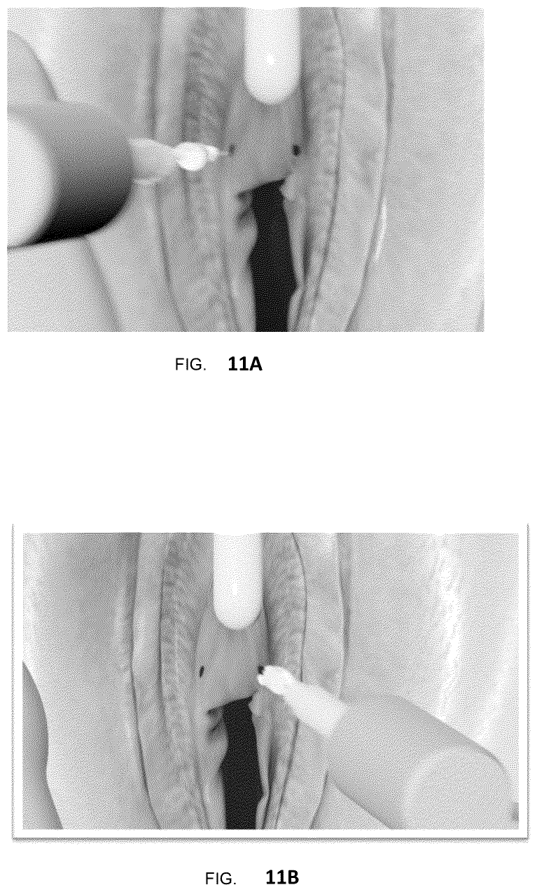

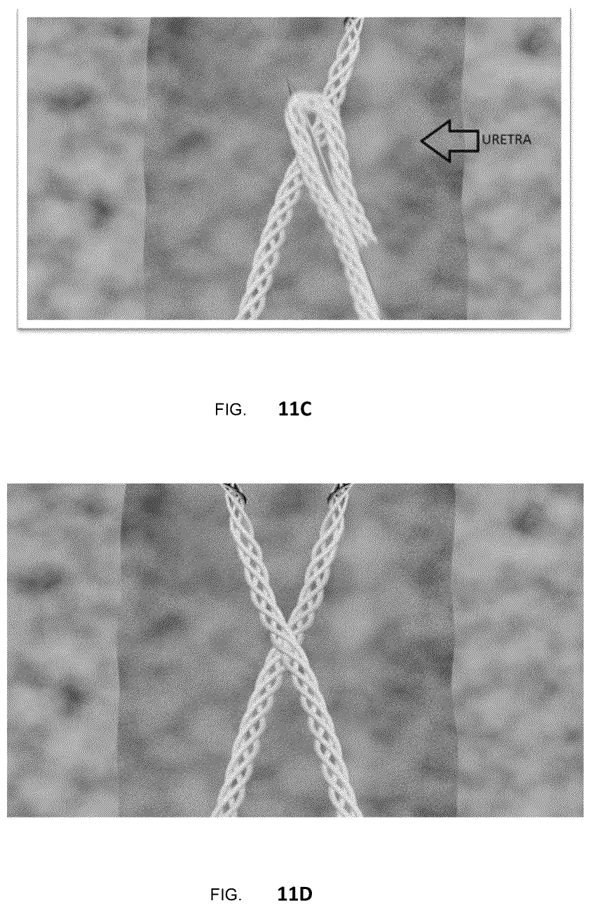

Once this is done, two entry holes are made at the base of the vestibule with a 19G needle, and after injection of 2 cc of 2% lidocaine with epinephrine (FIG. 10), a mesh thread is mounted on the thread holder and entered through one of the holes. Entry is perpendicular and at a 75° angle to the end of the cannula (FIGS. 11A and 11B). It is then withdrawn, and the thread will be placed and crossed in the suburethral space.

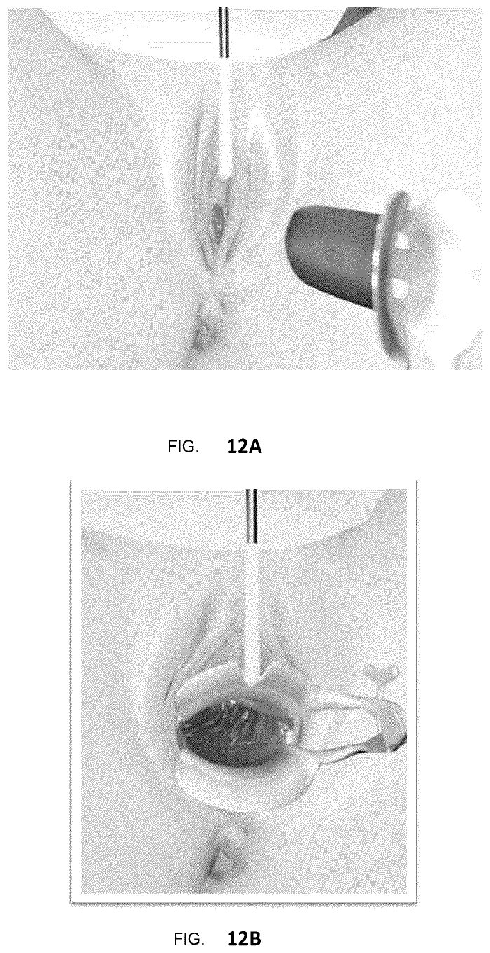

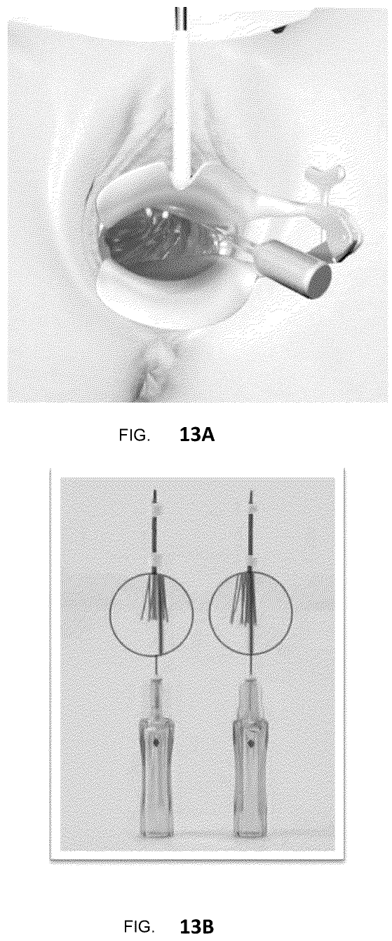

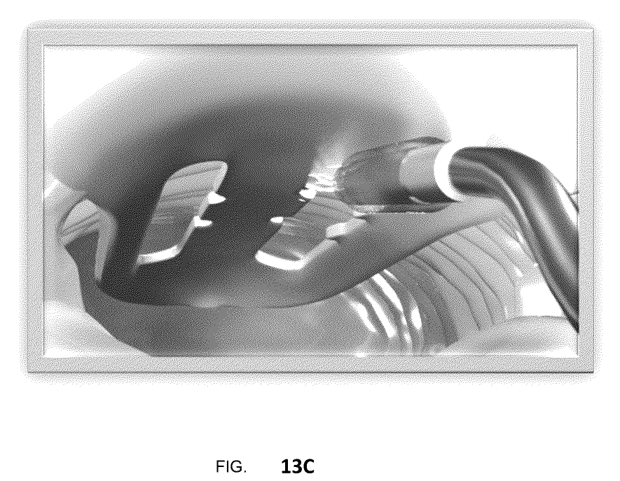

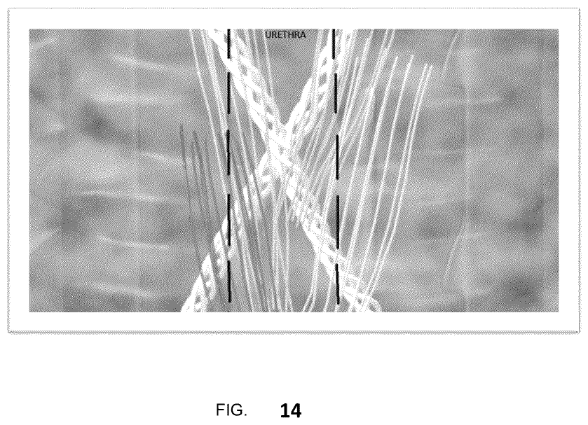

Repeating the same maneuver through the remaining orifice, the construction of the new support or floor of the urethra begins (FIGS. 11C and 11D). Next, the fenestrated speculum is placed and rotated until 12 o'clock (FIG. 12A), inserter 3 is fitted into the speculum notch and it is opened (FIG. 12B). On the front face of the speculum, and following the inclined slots of the windows, an entry hole is made for the needle mounted on the thread holder (FIG. 13A), to then introduce the assembled plury thread (FIG. 13B). on bayonet 1, and finally place it in the suburethral space (FIG. 13C).

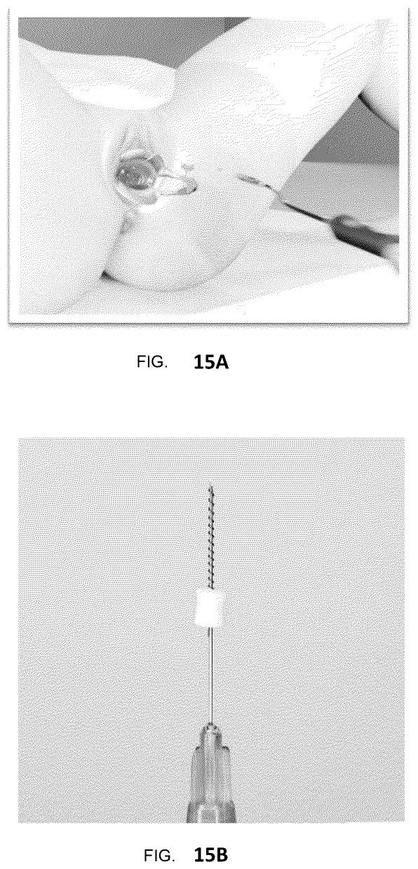

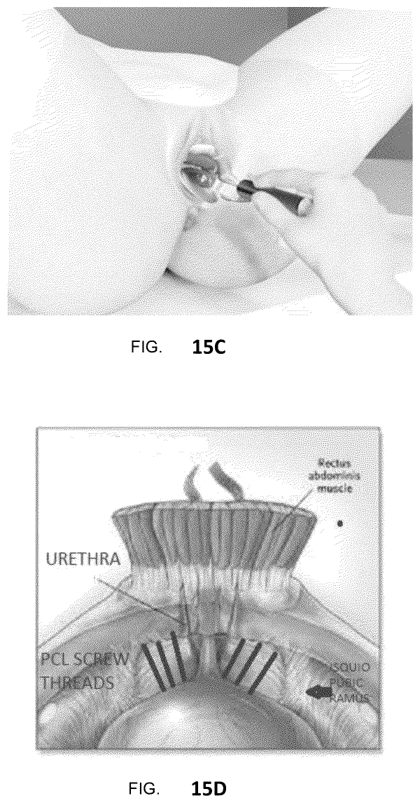

Finally, we proceed to apply the mono screw threads mounted on needles (FIG. 15B), with a blade laterally to the urethra and entering the urethrovaginal space through the speculum windows, with the help of bayonet 1 (FIGS. 15A and 15C). The threads are placed by rotating the speculum three times to each side and placing three threads per movement (FIG. 15D), and in such a way—with the sub urethral net previously placed, the mesh is completed, so ischio-pubian branch to branch and from the urethral meatus to the bladder, giving it a new sustenance or floor to the urethra throughout its journey.

What is claimed is:

1. A technique for the treatment of stress urinary incontinence in women, characterized by the placement of a network of reabsorbable threads in the sub urethral and urethro vaginal spaces.

2. The technique of claim 1 characterized by the use of polycaprolactone (PCL) and polydioxanone (PDO) threads, mesh threads, pluri yarns and mono screw threads, and their administration through the use of instruments or tools of appropriate design, whose patentability is also claimed.

3. The instruments or tools mentioned claim 2, consisting of a fenestrated speculum, an introducer element, a bayonet and a thread holder.

4. The fenestrated speculum of claim 3, characterized by presenting two fenestrations or lateral and longitudinal “windows”, 7 mm wide by 65 mm long, a central rib, on the inner edges of each window there are inclined grooves for the operator guide, and a grimace or notcha at 12 o'clock.

5. The introducer element of claim 3, consisting of a handle or clamp and a hook at its end, of appropriate shape and measurements for its entry into the urethra through and from the urethral meatus to the bladder.

6. The bayonet of claim 3, consisting of a handle and a rod in the shape of an italic “S”, which ends with a pointer adaptable to pressure to the rear part of the needle or thread-holding cannula.

7. The thread-holding instrument of claim 3, consisting of a cylindrical body and an adaptable pressure pointer, intended for handling and placing the mesh threads.

8. The technique of claim 1, characterized by the previous placement of the introducer through the urethral meatus, and elevation of the same for the lengthening of the elongation of the vulvar vestibule.

9. The technique of claim 1, which then provides for the catheterization of the urethra with the introducer (3) which includes the assembly of a bladder catheter-, the execution of two entrance holes at the base of the vestibule and the entrance through one of them with a mesh thread by using the thread holder (7), repeating the same operation on the remaining hole.

10. The technique of claim 3 characterized in that the fenestrated speculum is then placed and proceeds to its rotation until 12 o'clock, to then fit the introducer in the notch of the speculum; then it opens itself and, following the inclined grooves of the windows, we proceed to make an entry hole with the needle mounted on the thread holder to introduce the pluri thread mounted on the bayonet (6) and finally place it in the suburethral space, repeating the same maneuver in each of the inclined slits of the fenestrated speculum.

11. The technique of claim 1, the use of which ends with the application of monoscrew threads mounted on needles—with edge laterally to the urethra and entering the urethrovaginal space through the windows of the speculum, by using the bayonet (6) and the placement of the threads by rotating the speculum for three consecutive times to each side and for each movement.
